(19) United States
(12) Patent Application Publication
(10) Pub. No.: US 2026/0315842 A1
(43) Pub. Date: Oct. 15, 2026

(54) VEHICULAR CONTROL SYSTEM WITH ENHANCED FIRE SENSING AND LIMITING OF FIRE HAZARDS

(71) Applicant: Magna Electronics Inc., Auburn Hills, MI (US)

(72) Inventors: Ramallingeswara Rao P, Andhra Pradesh (IN); Nanubala Guru Siva Prasad, Karnataka (IN)

(21) Appl. No.: 19/466,786

(22) Filed: Feb. 2, 2026

Related U.S. Application Data
(60) Provisional application No. 63/760,227, filed on Feb. 19, 2025.

(51) Int. Cl. A62C 3/07; A62C 37/36; B60Q 9/00; G01N 33/00; H04W 4/90

(52) U.S. Cl. CPC A62C 3/07; A62C 37/36; B60Q 9/00; G01N 33/0062; H04W 4/90

(57) ABSTRACT

A vehicular alert system includes a sensor disposed at a compartment of a vehicle equipped with the vehicular alert system and sensing at least a portion of the compartment. A processor processes sensor data captured by the sensor to determine an indicator of a fire. Responsive to determining the indicator of a fire, the vehicular alert system determines a likelihood that a fire is present at the vehicle. Responsive to determining the likelihood that a fire is present at the vehicle, the vehicular alert system alerts an occupant of the vehicle.

### CROSS REFERENCE TO RELATED APPLICATION

The present application claims the filing benefits of U.S. provisional application Ser. No. 63/760,227, filed Feb. 19, 2025, which is hereby incorporated herein by reference in its entirety.

### FIELD OF THE INVENTION

The present invention relates generally to a vehicular alert system for a vehicle and, more particularly, to a vehicular alert system that utilizes one or more sensors at a vehicle.

### BACKGROUND OF THE INVENTION

Use of sensors in vehicle alert systems is common and known. Examples of such known systems are described in U.S. Pat. Nos. 5,949,331; 5,670,935 and/or 5,550,677, which are hereby incorporated herein by reference in their entireties.

### SUMMARY OF THE INVENTION

A vehicular alert system includes a sensor disposed at a compartment of a vehicle equipped with the vehicular alert system and sensing at least a portion of the compartment. The system includes an electronic control unit (ECU) comprising electronic circuitry and associated software. The electronic circuitry of the ECU includes a processor for processing sensor data captured by the sensor. Sensor data captured by the sensor is transferred to and is processed at the processor of the ECU, and the processor processes sensor data captured by the sensor to determine an indicator of a fire at the vehicle. Responsive to determining the indicator of a fire, the vehicular alert system determines a likelihood that a fire is present at the vehicle. Responsive to determining the likelihood that the fire is present at the vehicle, the vehicular alert system alerts an occupant of the vehicle.

These and other objects, advantages, purposes and features of the present invention will become apparent upon review of the following specification in conjunction with the drawings.

### BRIEF DESCRIPTION OF THE DRAWINGS

FIG. 1 is a plan view of a vehicle with an alert system that incorporates sensors; and

FIG. 2 is a block diagram of a vehicular alert system including integrated advanced smart firefighting.

### DESCRIPTION OF THE PREFERRED EMBODIMENTS

A vehicular alert system may alert a driver or an occupant of a vehicle of hazardous conditions determined by the vehicular alert system. In some examples, a hazard may arise from a condition of the vehicle itself. For example, the vehicular alert system may alert a driver of the vehicle of a failure of a system or component of the vehicle, such as an engine failure, a battery failure, an electrical system failure, low tire pressure, or any other condition that may negatively affect operation of the vehicle.

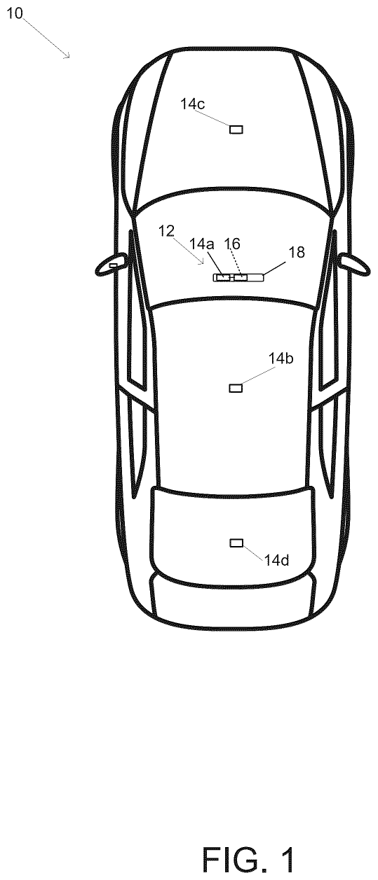

Referring now to the drawings and illustrative embodiments depicted therein, a vehicle 10 includes a vehicular alert system 12 that includes at least one sensor 14a, with the sensor 14a having a field of sensing of at least a portion of a component, a compartment and/or an area of the vehicle 10 (FIG. 1), such as a cabin (e.g., passenger compartment), an engine compartment, an engine, a catalytic converter, a turbocharger, a battery compartment, a battery, a trunk and/or storage compartment, an underbody of the vehicle, etc. The system 12 includes a control or electronic control unit (ECU) 16 that comprises electronic circuitry and associated software, with the electronic circuitry including a data processor or image processor that is operable to process sensor data captured by the sensor or sensors (and may process image data captured by other cameras at the vehicle, such as exterior viewing cameras disposed at locations so as to have respective fields of view exterior of the vehicle) and/or may provide an output to a display device for viewing by the driver of the vehicle. The output may be communicated to another system or ECU of the vehicle, or to a third party. Although shown in the illustrated embodiment as disposed at an interior rearview mirror assembly 18 of the vehicle 10, the sensor 14a and ECU 16 (and optionally one or more near infrared light emitters) may be disposed at any suitable position within the vehicle, such as at a dashboard, gauge cluster, windshield mounted electronics module, or the like. Sensor 14a and/or additional sensors 14b-14d may be disposed at a passenger compartment, an engine compartment, a storage compartment and/or a trunk, etc. The data transfer or signal communication from the sensor(s) 14a-14d to the ECU 16 may comprise any suitable data or communication link, such as a vehicle network bus or the like of the equipped vehicle 10.

Car accidents and operational failures involving a gasoline vehicle (e.g., internal combustion engine vehicle, an internal combustion vehicle, a diesel vehicle), an electric vehicle, a hydrogen vehicle, and/or a hybrid vehicle implicate different safety considerations. Gasoline vehicles (e.g., vehicles that include an internal combustion engine) are susceptible to fires (i.e., thermal events) due to the combustible nature of gasoline or diesel fuel. In internal combustion vehicles, high surface temperatures of exhaust manifolds, turbochargers, or catalytic converters may serve as ignition sources for leaking fluids. Furthermore, electrical shorts in a 12-volt wiring harness may ignite plastic insulation or interior materials. Fuel leaks, engine malfunctions, and accidents may cause rapid and intense flames. The presence of a fuel tank increases the risk of explosions. Similarly, hydrogen vehicles are susceptible to fires related to a pressurized fuel tank and the high volatility of hydrogen fuel. Hydrogen fires may present unique detection challenges, as hydrogen flames may burn with low radiant heat and may be nearly invisible to the naked eye in daylight conditions. Electric vehicles are also susceptible to fires due to high-voltage lithium-ion batteries. For example, issues including thermal runaway and electrical faults and/or failures may lead to fires. Thermal runaway in electric vehicles may result from separator failure, dendrite growth, physical damage to the battery pack, or overcharging events. Unlike internal combustion fires, battery fires may release toxic off-gases (e.g., hydrogen fluoride) and may be susceptible to reignition events hours or days after the initial suppression due to stranded energy within the battery cells. Hybrid vehicles may present a compounding of risks, containing both high-voltage electrical systems capable of arcing and combustible liquid fuels. Understanding the respective risks relating to different vehicle types helps in taking appropriate safety measures for both types of vehicles.

Thousands of highway vehicle fires are reported annually in the United States. The most common cause of vehicle fires is a fuel system leak. Such leaks may result from aged or cracked fuel lines, faulty fuel injectors, or loose connections caused by vehicle vibration over time. The second most common cause of vehicle fires is electrical system failure. Electrical failures may include short circuits, overloaded circuits, battery terminal corrosion leading to high resistance and heat generation or chafing of wire insulation against the vehicle chassis. Other common causes of vehicle fires include spilled fluids (e.g., power steering fluid, brake fluid, or engine oil), overheating engines, overheating catalytic converters, poor maintenance, and car crashes. Spilled fluids may ignite when in contact with hot surfaces. Overheating engines and overheating catalytic converters may cause fires due to excessive heat igniting fuel and/or vehicle components adjacent to the heat source. For example, a clogged catalytic converter may reach temperatures sufficient to ignite underbody debris or dry vegetation beneath the vehicle. Poor maintenance increases risk of mechanical failures such as seized bearings or dragging brakes that may lead to fires via friction-induced heat. Impacts from car crashes may result in fuel leaks and fires. Additionally, transporting flammable devices and goods or other volatile products, such as in a cargo area of a vehicle, may also increase the risk of a vehicle fire. In some examples, aftermarket modifications to the vehicle, such as improperly installed stereo systems or lighting, may introduce new electrical fire hazards.

Fire detection systems may include a variety of sensors, such as heat detectors, smoke detectors, flame detectors, and imaging devices. Heat detectors (e.g., thermometers, thermocouples, thermistors, or linear heat detection cables) detect high temperatures or rapid rates of temperature rise. Linear heat detection cables may be routed throughout an engine bay or battery pack to detect overheating at any point along the length of the cable. Smoke detectors detect the presence of airborne particulate matter (e.g., smoke) that may indicate the presence of a fire. These sensors may utilize ionization detection technology to identify flaming fires or photoelectric detection technology to identify smoldering fires. In some implementations, the system may utilize aspirating smoke detectors that actively draw air samples from the compartment for analysis. Flame detectors detect the presence of flames using infrared or ultraviolet sensors. For example, the flame detector may be an infrared camera or an ultraviolet camera configured to detect specific wavelengths of light emitted by burning hydrocarbons or hydrogen. Other imaging devices may also be used, including cameras that operate in the visible light spectrum to capture image data. An image processor may process the image data for shapes, colors, or differences in light intensity that may indicate the presence of a fire. In some examples, the system may utilize thermal imaging cameras (e.g., micro-bolometers) to identify hot spots or thermal gradients on vehicle components before a fire ignites. The system may also employ gas sensors configured to detect specific gases indicative of a thermal event, such as hydrocarbon vapors from a fuel leak, hydrogen gas from a fuel cell leak, or electrolyte vapors (e.g., carbonates, diethyl carbonate) vented from a lithium-ion battery cell prior to thermal runaway. Additionally, the system may incorporate acoustic sensors to detect sounds associated with thermal events, such as the hissing of a high-pressure leak, the popping of battery safety vents, or the crackling of combustion. Other types of detectors may also help identify when a fire is or may be present.

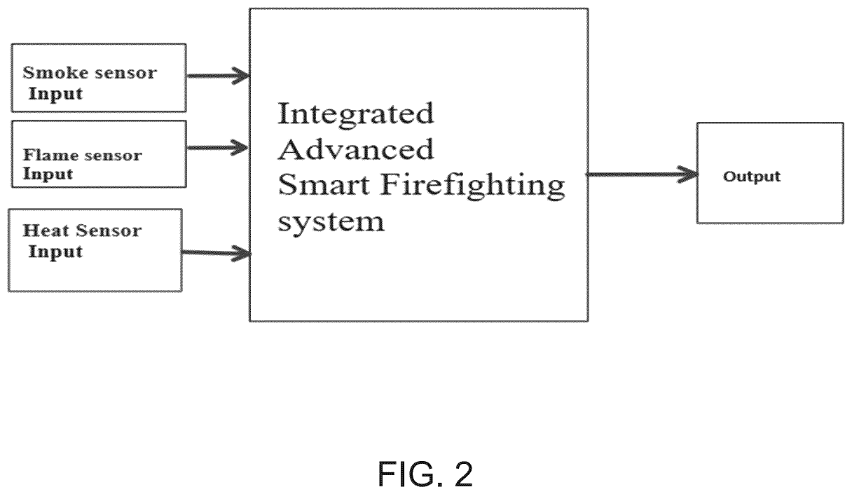

FIG. 2 illustrates a block diagram of a vehicular alert system 12 including integrated advanced smart firefighting (e.g., an integrated advanced firefighting system, or a smart firefighting system). The integrated advanced smart firefighting system may be embodied within the electronic control unit (ECU) 16, or may be distributed across multiple controllers within the vehicle 10. The smart firefighting system may receive input from one or more fire detecting devices or one or more sensors 14a-14d, the inputs comprising sensor data. The sensor data may be communicated via a vehicle network, such as a Controller Area Network (CAN) bus, a Local Interconnect Network (LIN) bus, an automotive Ethernet connection, or a wireless protocol. For example, the smart firefighting system may receive sensor data from a smoke sensor (e.g., an ionization or photoelectric smoke detector, or an aspirating smoke detector system that draws air samples from the battery compartment or cabin), a flame sensor (e.g., a UV or IR flame sensor or detector, or a multi-spectrum infrared detector configured to distinguish between flame emissions and non-fire radiation sources), and/or a heat sensor (e.g., a heat detector, a temperature sensor, or thermometer, such as a thermocouple, a thermistor, or a resistance temperature detector). Based at least in part on the inputs of the sensors, the smart firefighting system determines the presence of a fire or other thermal event. In some implementations, the ECU 16 utilizes a sensor fusion algorithm to correlate data from the disparate sensor types to increase detection accuracy. The smart firefighting system may determine presence of a fire or thermal event based on determining the presence of one or more indicators of a fire, including smoldering, smoke, heat, flames, particulate matter, infrared light, ultraviolet light, visible light, carbon dioxide, carbon monoxide, hydrocarbon vapors, hydrogen gas, or rapid pressure changes within a compartment.

The smart firefighting system may receive sensor data from other types of sensors including cameras (e.g., thermal imaging cameras, red-green-blue (RGB) cameras, or near-infrared cameras), a carbon monoxide detector, a carbon dioxide detector, a volatile organic compound (VOC) sensor, etc. The ECU 16 may employ computer vision algorithms, such as object detection neural networks or edge detection logic, to analyze image data for visual signatures of fire, such as the flickering motion of flames or the volumetric expansion of smoke plumes. Based on the sensor data, the smart firefighting system may produce an output. The output may indicate that a fire or other thermal event is present, that a fire is not present, that a fire may be present, or that there is an elevated risk of a fire, etc. In some embodiments, the output may indicate that a fire is or may be present based on the indication of a single sensor. For example, where sensor data from the smoke sensor indicates that smoke is present, but sensor data from the flame sensor and the heat sensor indicate that a fire is not present, the smart firefighting system may provide an output that a fire is or may be present. In other embodiments, the output of the smart firefighting system may indicate that a fire is or may be present only if more than one sensor indicates a fire is or may be present (e.g., using AND logic or a voting scheme). This multi-sensor confirmation strategy may reduce the incidence of false alarms caused by non-hazardous events, such as cigarette smoke, exhaust steam, or roadway dust. In one example, where sensor data from the smoke sensor indicates that smoke is present, but sensor data from the flame sensor and the heat sensor indicate that a fire is not present, the smart firefighting system may determine a false positive detection, and the output may indicate that a fire is not present. In a second example, where sensor data from the smoke sensor indicates that smoke is present, and the flame sensor indicates that flames are present, but the heat sensor does not indicate that a fire is present, the system may determine a fire is or may be present, and the output may indicate that a fire is or may be present. In some examples, the system 12 may utilize a confidence score derived from the multiple sensors. If the confidence score exceeds a predetermined verification threshold, the system 12 confirms the presence of the fire.

In further embodiments, the output of the smart firefighting system may be a determination that the vehicle 10 is at a heightened risk of a fire. This determination may be a predictive assessment indicating that a thermal event is imminent or highly probable if current conditions persist. In one example, the smart firefighting system may determine that a component, a compartment, or an area of the vehicle is at a heightened risk of a fire, based at least in part on temperature data from the heat sensor exceeding a threshold temperature. The threshold temperature may be a static value or a dynamic value based on the operating state of the vehicle 10 (e.g., a higher threshold may apply when the engine is under high load compared to when the vehicle is parked). The smart firefighting system may apply different threshold temperatures based on different heat sensor locations about the vehicle. For example, a sensor 14c in the engine compartment may have a higher temperature threshold than a sensor 14d in the trunk or a sensor 14b in the passenger cabin. In another example, the smart firefighting system may determine that the vehicle is at a heightened risk of a fire based at least in part on data regarding an ambient environment of the vehicle, either alone or in combination with inputs from the sensors. For example, the system may determine that the vehicle is at a heightened risk of fire based on an ambient temperature of the environment of the vehicle or based on location data (e.g., GPS data) indicating the vehicle 10 is in a region experiencing wildfires or extreme heat. Optionally, the system may determine that the vehicle is at a heightened risk of fire based at least in part on the ambient temperature of the environment of the vehicle (or other environmental conditions such as humidity or barometric pressure) in combination with sensor data from the heat sensor. The system 12 may also analyze a rate of change (e.g., the derivative) of the temperature data; a rapid spike in temperature, even if below the absolute maximum threshold, may trigger a heightened risk determination.

The output may indicate a likelihood that a fire is present, and the vehicular alert system 12 may alert an occupant of the vehicle 10 when the likelihood that a fire is present exceeds a threshold value. For example, the vehicular alert system may alert an occupant of the vehicle when the likelihood that a fire is present exceeds 1%, 5%, 10%, 20% or 25%. In some implementations, the threshold value may be adjustable based on user preferences or dealer settings. The smart firefighting system may determine the likelihood that a fire is present based on applying a sliding scale to sensor data inputs from each of the sensors 14a-14d. In one example, the percentage likelihood of a fire may increase as the temperature of the temperature sensor data increases. For instance, the ECU 16 may reference a look-up table stored in a memory that correlates specific temperature ranges with specific probability percentages. In another example, the percentage likelihood of a fire may increase as the parts-per-billion of particulate matter of the smoke sensor data increases. In still another example, the percentage likelihood of a fire may increase as the intensity of infrared light of the flame sensor data increases. Additionally or alternatively, the smart firefighting system may determine the likelihood that a fire is present based on aggregating the sliding-scale sensor data input from each of the sensors. For example, the smart firefighting system may determine the percentage likelihood of a fire by combining the individual percentage likelihood of a fire as indicated by sensor data of each individual sensor as described above. The ECU 16 may utilize a Bayesian probability model, a fuzzy logic algorithm, or a neural network to process the aggregated sensor data and calculate the final likelihood metric. Accordingly, where a first heat sensor (e.g., a temperature sensor) indicates a 5% likelihood that a fire is present and a second heat sensor indicates a 10% likelihood that a fire is present, the smart firefighting system may determine a 7.5% likelihood that a fire is present. Aggregating the individual percentage likelihoods of a fire may include adding the percentages together, multiplying the percentages, averaging and/or weighting the percentages, and/or other means of combining the percentages. For example, the ECU 16 may assign a higher weight to the smoke sensor data than the heat sensor data when the vehicle 10 is in a cold start condition, or may assign a higher weight to the flame sensor data when the vehicle is moving at highway speeds where smoke might be rapidly dispersed.

The output may further include information regarding which sensor(s) 14a-14d indicate the presence of a fire. The output may also include information regarding a likely location of the fire, a likely cause and/or source of the fire, and/or a type of fire (e.g., an electrical fire, a gasoline fire, a battery electrolyte fire, a metallic fire, etc.). The ECU 16 may identify the likely location of the fire by triangulating data from multiple sensors or by identifying which specific zonal sensor (e.g., engine compartment sensor 14c vs. trunk sensor 14d) triggered the alert. To determine the likely cause, the ECU 16 may correlate the fire detection time with On-Board Diagnostic (OBD) trouble codes, such as a “cylinder misfire” code or a “battery cell voltage deviation” code. In examples where the smart firefighting system output includes the type of fire, the system may determine the type of fire based at least in part on a temperature of the fire, a location of the fire, the type of propulsion system of the vehicle equipped with the smart firefighting system, data or faults from one or more sensors, etc. For example, the system may analyze the spectral signature of the flames captured by an imaging sensor; a blue flame may indicate a hydrogen fire, whereas an orange, sooty flame may indicate a hydrocarbon fuel fire. Additionally, the system 12 may record this output data to a non-volatile memory or an Event Data Recorder (EDR) to aid in post-incident forensic analysis.

When a fire is detected in the vehicle 10, the smart firefighting system may activate to suppress the fire. The system may use one or more sensors 14a-14d including heat sensors, smoke sensors, and/or infrared sensors to detect indicators of a fire. Based on sensor data from the one or more sensors, the system may determine presence of a fire (or conditions conducive to a fire or other thermal event). Responsive to determining presence of a fire, the system may automatically activate to release (i.e., deploy) a fire suppression agent (i.e., a hazard extinguishing agent). The deployment mechanism may include opening electromechanical valves, triggering pyrotechnic actuators to puncture pressurized canisters, or activating aerosol generators. The fire suppression agent may be water, a liquid chemical, a dry chemical (e.g., monoammonium phosphate), carbon dioxide, an inert gas, a clean agent (e.g., a fluorinated ketone or a heptafluoropropane), etc. In some embodiments, the smart firefighting system may release the fire suppression agent locally at the determined location of the fire. For example, if the fire is detected within a specific module of a high-voltage battery pack, the system may direct the suppression agent solely to that module via a dedicated piping network or manifold, thereby minimizing damage to adjacent non-affected modules. The smart firefighting system may selectively deploy one of a plurality of fire suppression agents based on determining the type of fire, the likely cause and/or source of the fire, the temperature of the fire, etc. For instance, the system may select a non-conductive clean agent for an electrical fire in the dashboard to prevent short circuits, while selecting an aqueous film-forming foam (AFFF) for a gasoline fire in the engine compartment. Optionally, the system may provide a means for manual activation of the fire suppression agent by an occupant of the vehicle, such as via a button (e.g., a “break-glass” switch or a covered toggle switch) or auditory command. The system 12 may require a secondary confirmation or may enforce safety interlocks (e.g., ensuring the vehicle speed is below a threshold) prior to permitting manual activation. By deploying the fire suppression agent, the smart firefighting system assists in preventing the fire from spreading, causing damage to the vehicle or its surroundings, or causing injury to occupants or bystanders proximate to the vehicle. The smart firefighting system may increase passenger safety and decrease damage to the vehicle.

The smart firefighting system may evaluate sensor data in real time (e.g., with processing cycles occurring at millisecond intervals) and, responsive to determinations of the system based on the sensor data, activate firefighting measures and/or fire suppression measures. If fire, smoke, heat, or other indicators of a fire are detected, the system may automatically engage the firefighting mechanism to extinguish flames and prevent smoke from spreading to various parts of the car. To ensure fire safety, the smart firefighting system may provide immediate audible, visual, or haptic alerts. For example, the system may provide audio or visual alerts at a dashboard and/or infotainment system of the vehicle. Visual alerts may include illuminating specific warning icons on an instrument cluster, flashing interior ambient lighting in a specific color code (e.g., red), or projecting a warning onto a windshield via a Head-Up Display (HUD). Haptic alerts may include vibrating a steering wheel or a driver's seat. In some examples, the alert may indicate the determined location of the fire, the determined cause of the fire, and/or the determined type of fire. The immediate alerts may enable a driver of the vehicle to react promptly to a potential fire. In some implementations, the system may communicate with a mobile device of the user via a short-range wireless protocol (e.g., Bluetooth, Wi-Fi, Near Field Communication) or a cellular network to provide a remote alert, which may be particularly beneficial if the user is away from the vehicle. In some examples, the smart firefighting system may notify first responders or a third party upon determining presence of a fire via a telematics system (e.g., an emergency call or eCall system). Furthermore, the system may utilize Vehicle-to-Vehicle (V2V) or Vehicle-to-Infrastructure (V2I) communication protocols to broadcast a hazard warning to nearby vehicles or traffic management centers, thereby advising other motorists to maintain a safe distance. Implementing a smart firefighting system may enhance vehicle safety and protect the driver, occupants, or bystanders proximate to the vehicle from a potential fire hazard.

The smart firefighting system may place other systems of the equipped vehicle into a failure mode (i.e., a protective state or a reduced performance state to prevent damage to the vehicle or reduce risk to occupants of the vehicle) to reduce or prevent spreading of the fire. For example, the system may instruct a body control module (BCM) to unlock all vehicle doors and/or lower the windows to facilitate occupant egress and allow smoke to ventilate from the cabin. The system may also automatically activate exterior hazard lights and the horn to attract attention. In some examples where the vehicle includes an internal combustion engine, the smart firefighting system may deactivate one or more fuel pumps of the vehicle or close an air intake throttle to starve the fire of oxygen. In other examples where the vehicle includes an electric battery, the smart firefighting system may electrically isolate electrical components of the vehicle, such as the battery, electrical motors, or electrical inverters (e.g., by opening high-voltage contactors or firing a pyrotechnic safety switch). The system may also adjust the operation of a heating, ventilation, and air conditioning (HVAC) system. For example, the system may shut down HVAC blowers to prevent the circulation of smoke into the passenger compartment, or alternatively, reverse the airflow to exhaust smoke.

Sensors of the smart firefighting system may be implemented at a trunk, cargo space, passenger compartment, engine compartment, wheel wells, underbody shields, charging ports, or other location of the vehicle where potential fire hazards or sources exist. The smart firefighting system may be implemented in personal or commercial vehicles (e.g., semi-trucks, delivery vans, buses, taxis). The system may be particularly adapted for autonomous vehicles (e.g., robotaxis, autonomous delivery pods) where a human driver is not present to monitor for signs of fire. Although the smart firefighting system is herein disclosed as integrated with a vehicle, the smart firefighting system may be integrated in any type of vehicle (e.g., a nautical vehicle, an amphibious vehicle, an aeronautical vehicle such as an airplane or helicopter, an aerospace vehicle, an unmanned aerial vehicle (UAV) or drone, etc.). The smart firefighting system may also be integrated into a residential building, a commercial building, or any manmade structure (e.g., a warehouse, a data center server room, a parking garage). The smart firefighting system may be integrated into an outdoor environment. For example, the sensors may be distributed at various points (e.g., outposts) in an outdoor environment (e.g., a forest, a national park, an oil field, an industrial plant), and the sensors communicate sensor data to a device located either locally or remotely to process the data and determine the presence of a fire. In a residential or commercial setting, the system may integrate with a broader building automation system, allowing the sensors to trigger building-wide alarms, activate sprinkler systems, or shut down central air handling units.

The camera or sensor may comprise any suitable camera or sensor. Optionally, the camera may comprise a “smart camera” that includes the imaging sensor array and associated circuitry and image processing circuitry and electrical connectors and the like as part of a camera module, such as by utilizing aspects of the vision systems described in U.S. Pat. No. 10,099,614 and/or 10,071,687, which are hereby incorporated herein by reference in their entireties.

The imaging device and control and image processor and any associated illumination source, if applicable, may comprise any suitable components, and may utilize aspects of the cameras (such as various imaging sensors or imaging array sensors or cameras or the like, such as a CMOS imaging array sensor, a CCD sensor or other sensors or the like) and vision systems described in U.S. Pat. Nos. 5,760,962; 5,715,093; 6,922,292; 6,757,109; 6,717,610; 6,590,719; 6,201,642; 5,796,094; 6,559,435; 6,831,261; 6,822,563; 6,946,978; 7,720,580; 8,542,451; 7,965,336; 7,480,149; 5,877,897; 6,498,620; 5,670,935; 5,796,094; 6,396,397; 6,806,452; 6,690,268; 7,005,974; 7,937,667; 7,123,168; 7,004,606; 6,946,978; 7,038,577; 6,353,392; 6,320,176; 6,313,454 and/or 6,824,281, and/or International Publication Nos. WO 2009/036176; WO 2009/046268; WO 2010/099416; WO 2011/028686 and/or WO 2013/016409, and/or U.S. Publication Nos. US 2010-0020170 and/or US-2009-0244361, which are all hereby incorporated herein by reference in their entireties.

Changes and modifications in the specifically described embodiments can be carried out without departing from the principles of the invention, which is intended to be limited only by the scope of the appended claims, as interpreted according to the principles of patent law including the doctrine of equivalents.

1. A vehicular alert system, the vehicular alert system comprising:
a sensor disposed at a compartment of a vehicle equipped with the vehicular alert system and sensing at least a portion of the compartment;
an electronic control unit (ECU) comprising electronic circuitry and associated software;
wherein the electronic circuitry of the ECU comprises a processor for processing sensor data captured by the sensor;
wherein sensor data captured by the sensor is transferred to and is processed at the processor of the ECU;
wherein the processor processes sensor data captured by the sensor to determine an indicator of a fire;
wherein, responsive to determining the indicator of a fire, the vehicular alert system determines a likelihood that a fire is present at the vehicle; and
wherein, responsive to determining the likelihood that a fire is present at the vehicle, the vehicular alert system alerts an occupant of the vehicle.

2. The vehicular alert system of claim 1, wherein the indicator of a fire comprises at least one selected from the group consisting of (i) particulate matter, (ii) heat, (iii) infrared light, (iv) ultraviolet light, (v) visible light, (vi) carbon dioxide, and (vii) carbon monoxide.

3. The vehicular alert system of claim 1, wherein the sensor comprises at least one selected from the group consisting of (i) a smoke sensor, (ii) a flame sensor, (iii) a heat sensor, (iv) a camera, (v) a carbon dioxide sensor, and (vi) a carbon monoxide sensor.

4. The vehicular alert system of claim 1, further comprising a plurality of sensors.

5. The vehicular alert system of claim 1, wherein the vehicular alert system determines that the likelihood that a fire is present at the vehicle by determining the likelihood that a fire is present at the vehicle exceeds a threshold value.

6. The vehicular alert system of claim 1, wherein the vehicular alert system alerts the occupant of the vehicle via at least one selected from the group consisting of (i) an audible alert, (ii) a visual alert and (iii) a haptic alert.

7. The vehicular alert system of claim 1, wherein the vehicular alert system, responsive to determining the likelihood that a fire is present at the vehicle, places another system of the vehicle into a failure mode.

8. The vehicular alert system of claim 1, wherein the vehicular alert system, responsive to determining the likelihood that a fire is present at the vehicle, alerts a third party.

9. The vehicular alert system of claim 1, wherein the vehicular alert system, responsive to determining the likelihood that a fire is present at the vehicle, releases at least one fire suppression agent.

10. The vehicular alert system of claim 9, wherein the at least one fire suppression agent comprises at least one selected from the group consisting of (i) water, (ii) a liquid chemical, (iii) a dry chemical, (iv) carbon dioxide, (v) an inert gas (vi) a clean agent.

11. The vehicular alert system of claim 9, wherein the vehicular alert system releases the fire suppression agent responsive to an input from the occupant of the vehicle.

12. The vehicular alert system of claim 9, wherein the at least one fire suppression agent includes a plurality of fire suppression agents.

13. The vehicular alert system of claim 12, wherein the vehicular alert system selectively releases one of the plurality of fire suppression agents.

14. The vehicular alert system of claim 1, wherein the compartment comprises one selected from the group consisting of (i) a cabin of the vehicle, (ii) an engine compartment of the vehicle, (iii) a battery compartment of the vehicle, (iv) an underbody of the vehicle, and (v) a storage compartment of the vehicle.

15. A method of alerting an occupant of a vehicle equipped with a vehicular alert system, the method comprising:
sensing, by a sensor disposed at a compartment of the vehicle, at least a portion of the compartment;
transferring sensor data captured by the sensor to a processor of an electronic control unit (ECU) comprising electronic circuitry and associated software;
processing, by the processor, the sensor data captured by the sensor to determine an indicator of a fire;
determining, responsive to determining the indicator of a fire, a likelihood that a fire is present at the vehicle; and
alerting, responsive to determining the likelihood that a fire is present at the vehicle, the occupant of the vehicle.

16. The method of claim 15, wherein the indicator of a fire comprises at least one selected from the group consisting of (i) particulate matter, (ii) heat, (iii) infrared light, (iv) ultraviolet light, (v) visible light, (vi) carbon dioxide, and (vii) carbon monoxide.

17. The method of claim 15, wherein the sensor comprises at least one selected from the group consisting of (i) a smoke sensor, (ii) a flame sensor, (iii) a heat sensor, (iv) a camera, (v) a carbon dioxide sensor, and (vi) a carbon monoxide sensor.

18. The method of claim 15, wherein the sensor comprises a plurality of sensors.

19. The method of claim 15, wherein determining the likelihood that a fire is present at the vehicle comprises determining that the likelihood that a fire is present at the vehicle exceeds a threshold value.

20. The method of claim 15, wherein alerting the occupant of the vehicle comprises alerting via at least one selected from the group consisting of (i) an audible alert, (ii) a visual alert and (iii) a haptic alert.
